(19) United States
(12) Patent Application Publication
(10) Pub. No.: US 2009/0214594 A1
(43) Pub. Date: Aug. 27, 2009

(54) Prevention and Treatment of Otitis Media with Non-Pathogenic Bacterial Strains

(75) Inventors: Marie-Claire Fichot, Blonay (CH); Harald Bruessow, La Tour De Peilz (CH)

Correspondence Address: K&L Gates LLP, P.O. Box 1135, CHICAGO, IL, 60690, US

(73) Assignee: NESTEC S.A., Vevey (CH)

(21) Appl. No.: 12/302,582

(22) PCT Filed: Jun. 12, 2007
(86) PCT No.: PCT/EP2007/055741

§ 371 Date: Nov. 26, 2008

(30) Foreign Application Priority Data
Jun. 13, 2006 (EP) 06115410.0

(51) Int. Cl. A61K 39/09; A61K 39/02

(52) U.S. Cl. 424/244.1; 424/234.1

(57) ABSTRACT

This invention relates to a composition comprising a bacterial strain capable of boosting the systemic immune response, a bacterial strain capable of exerting bacteriostatic effects on pathogens associated with development of otitis media such as Streptococcus pneumoniae, untypeable Haemophilus influenzae and Moraxella catarrhalis and a bacterial strain capable of exerting bacteriocidal effects on such pathogens. The invention further extends to the use of such a composition in the prevention or treatment of otitis media.

### FIELD OF THE INVENTION

This invention relates to the prevention and treatment of otitis media, particularly in infants and small children.

### BACKGROUND OF THE INVENTION

Infections of the respiratory tract are very common, particularly in infants and small children. For example, in the first year of life, an infant will often experience from three to six such infections. Such infections may be of bacterial or viral origin. Examples of viral infections of the respiratory tract include the common cold, influenza and respiratory syncytial virus. Examples of bacterial infections of the respiratory tract include pneumonia and otitis media.

Frequent respiratory tract infections are often associated with acute otitis media. This is an infection of the middle ear in which the Eustachian tube which connects the cavity of the middle ear with the external environment via the mouth becomes inflamed and then blocked trapping bacteria in the middle ear. The middle ear cavity also becomes inflamed with a build up of fluid leading to increased pressure which is experienced by the patient as pain due to the inability to equalise pressure between the middle ear and the external environment via the Eustachian tube as in healthy subjects. In severe cases, the tympanic membrane may burst under pressure allowing the infected liquid to reach the inner ear. This is a potentially dangerous situation which can lead to permanently impaired hearing if left untreated.

50% of children will have had at least one episode of acute otitis media in the first year of life and 35% of children between one and three years of age have recurrent episodes of acute otitis media. This in turn may lead to the development of a condition called glue ear in which the fluid does not completely drain from the middle ear between bouts of infection. If this condition becomes established, surgical intervention may be necessary.

Acute otitis media appears to be linked with the activity of pathogenic bacteria commonly found in the indigenous microbiota of the naso-pharyngeal cavity. Quantitatively, the most important pathogens are Streptococcus pneumoniae (35% of cases), untypeable Haemophilus influenzae (30% of cases) and Moraxella catarrhalis (10% of cases). For this reason, acute otitis media is commonly treated by the administration of antibiotics especially in infants. Indeed, antibiotics are prescribed more frequently for treatment of otitis media than for any other illness in infancy. This has inevitably led to the development of resistance to the commonly prescribed antibiotics in the bacterial strains associated with otitis media. For example, it is thought that at least 20% of S. pneumoniae strains are resistant to penicillins and cephalosporins. Similarly, at least 30% of H. influenzae strains and the majority of M. catarrhalis strains have developed antibiotic resistance. This frequency of prescription is at least in part due to the pain experienced by infants and young children suffering from otitis media to which they react by prolonged crying which parents and other care givers are very anxious to relieve. There is thus clearly a need for alternative methods to decrease the incidence of this painful and potentially serious condition in infants and young children.

Various alternative therapies have already been proposed. For example, in WO 97/17089 it is proposed to use a so-called immune milk preparation for the prevention of otitis media. This preparation contains anti-otitis immunoglobulins of the IgG type obtained from bovine colostrum to complement the passive immune defense.

Various bacterial strains have also been proposed for prevention/treatment of otitis media. In a recent clinical trial, inhibitory alpha hemolytic streptococci were sprayed into the noses of children with acute otitis media. The strains used were Streptococcus mitis, Streptococcus sanguis and Streptococcus oralis. The children treated in this way had less episodes of acute otitis media (Roos et al, Effect of recolonisation with “interfering alpha streptococci” on recurrences of acute and secretory otitis media in children: randomised placebo controlled trial, BMJ 322:210-212). However, the bacterial strains used in this trial are also recognised human pathogens implicated in conditions such as endocarditis and lung infections.

WO 2004/072272 proposes the use of a specific strain of Streptococcus salivarius in the prevention and treatment of otitis media. This strain is stated to be a bacteriocin producing strain which is non-pathogenic. It may be administered intranasally, by inhalation via the mouth or in the form of lozenges or capsules. Preferably, the strain is administered after an initial treatment with an antibiotic or other anti-microbial agent.

WO 2006/007526 describes a study of 81 infants in Finland in which the incidence of otitis media in infants fed a combination of a Lactobacillus rhamnosus strain and a Bifidobacterium lactis strain in infant formula was compared with that in a control group fed no probiotics. It was found that supplementation with these probiotics, both of which have a long history of use in human food products, decreased the risk of early acute otitis media by comparison with the unsupplemented group. This effect was associated with an overall reduction in the number of infections in the supplemented group and it is hypothesised that this effect was due to the systemic immunostimulatory effects of these strains.

From the foregoing, it may be seen that there remains a need for an effective method for the prevention and treatment of acute otitis media which does not rely on the use of antibiotics and which may be conveniently and safely administered.

### SUMMARY OF THE INVENTION

The present inventors have surprisingly found that the co-administration of bacterial strain(s) with complementary properties is particularly effective in the prevention and treatment of otitis media.

Accordingly, in a first aspect, the present invention provides a composition suitable for use in the prevention or treatment of otitis media comprising a non-pathogenic bacterial strain capable of boosting the systemic immune response, a non-pathogenic bacterial strain capable of exerting bacteriostatic effects on pathogens associated with development of otitis media and a non-pathogenic bacterial strain capable of exerting bacteriocidal effects on pathogens associated with development of otitis media.

In a second aspect, the present invention provides the use of a non-pathogenic bacterial strain capable of boosting the systemic immune response, a non-pathogenic bacterial strain capable of exerting bacteriostatic effects on pathogens associated with development of otitis media and a non-pathogenic bacterial strain capable of exerting bacteriocidal effects on pathogens associated with development of otitis media in the manufacture of a composition for the prevention or treatment of otitis media.

The invention further extends to a method for the prevention or treatment of otitis media which comprises administering to an individual in need thereof a therapeutic amount of a non-pathogenic bacterial strain capable of boosting the systemic immune response, a non-pathogenic bacterial strain capable of exerting bacteriostatic effects on pathogens associated with development of otitis media and a non-pathogenic bacterial strain capable of exerting bacteriocidal effects on pathogens associated with development of otitis media.

Without wishing to be bound by theory, the inventors believe that the efficacy of bacterial strain(s) as described above in the prevention and treatment of otitis media may be associated with pathology of the disease. It is thought that the mere presence of the pathogens S. pneumoniae, H. influenzae and M. catarrhalis will not inevitably result in the occurrence of acute otitis media. Rather it is thought that there must first be a primary viral infection of the upper respiratory tract which in some way not completely understood but probably related to the systemic immune response disturbs the microbiota of the nasopharynx allowing the pathogenic bacteria to colonise the mucosa and provoke an attack of otitis media. It is possible that simultaneously boosting the systemic immune response and the numbers of non-pathogenic bacteriostatic and bacteriocidal bacteria in the nasopharyngeal cavity may efficiently discourage colonisation of the nasopharyngeal mucosa by the pathogenic bacteria associated with the development of otitis media.

### DETAILED DESCRIPTION OF THE INVENTION

In the present specification, the following words are given a definition that must be taken into account when reading and interpreting the description, examples and claims.

The following definitions appear in Article 1.2 of the European Commission Directive 91/321/EEC of 14 May 1991 on infant formulae and follow-on formulae and are adopted in the present specification:—

“infant”: child under the age of 12 months;

“infant formulae”: foodstuffs intended for particular nutritional use by infants during the first four to six months of life and satisfying by themselves the nutritional requirements of this category of persons.

“follow-on formulae”: foodstuffs intended for particular nutritional use by infants aged over four months and constituting the principal liquid element in a progressively diversified diet of this category of persons.

“growing up milk” means a milk based beverage adapted for the specific nutritional needs of young children;

“non-pathogenic bacterial strain” means a bacterial strain which is not a recognised human pathogen;

“pathogens associated with development of otitis media” means one or more of Streptococcus pneumoniae, untypeable Haemophilus influenzae and Moraxella catarrhalis.

“young child” means a child between the age of one and six years.

All percentages are by weight unless otherwise stated.

It will be understood that the bacterial strain(s) used in the present invention must be live at the point of consumption. For this reason, the quantities of the strains are expressed in terms of colony forming units (cfu).

The bacterial strain capable of boosting the systemic immune response is preferably Lactobacillus rhamnosus ATCC 53103 or Lactobacillus rhamnosus CGMCC 1.3724. Lactobacillus rhamnosus ATCC 53103 may be obtained commercially from Valio Oy of Finland under the trade mark LGG®.

A bacteriostatic effect may be exhibited by strains which are effective producers of lactic acid. When these bacteria are orally administered, they produce lactic acid in quantities sufficient to exert an appreciable local bacteriostatic effect in the vicinity of the nasopharyngeal mucosa, thus discouraging colonization by pathogenic bacteria. One example of such bacteria is Streptococcus thermophilus. A suitable strain of S. thermophilus is that sold under the trade mark TH4 by Christian Hansen of Denmark.

Some bacterial strains are capable of both bacteriostatic and bacteriocidal activity. Such strains are preferred for use in the present invention because they can serve as both the bacterial strain capable of exerting bacteriostatic effects and the bacterial strain capable of exerting bacteriocidal effects, thus simplifying manufacturing complexity. Examples of such strains include Micrococcus varians MCV8 and Streptococcus salivarius DSM 13084 and DSM 13085. S salivarius DSM 13084 may be obtained commercially from BLIS Technologies Limited of New Zealand under the designation K12.

The strains may be administered using the same carrier or may be administered sequentially.

Preferably, the composition is a nutritional composition which is consumed as a liquid and is suitable for consumption by infants and young children. The composition may be a nutritionally complete formula such as an infant formula, a follow-on formula or a growing up milk. Alternatively for the older end of the target group of infants and young children, the composition may be a juice drink or other chilled or shelf stable beverage or a soup, for example.

Preferably a nutritional composition according to the invention contains from 10⁴ to 10¹² cfu/g of composition (dry weight) of each strain.

A nutritional composition according to the invention preferably further contains at least one prebiotic in an amount of 0.3 to 6%. A prebiotic is a non-digestible food ingredient that beneficially affects the host by selectively stimulating the growth and/or activity of one or a limited number of bacteria in the colon, and thus improves host health. Such ingredients are non-digestible in the sense that they are not broken down and absorbed in the stomach or small intestine and thus pass intact to the colon where they are selectively fermented by the beneficial bacteria. Examples of prebiotics include certain oligosaccharides, such as fructooligosaccharides (FOS) and galactooligosaccharides (GOS). A combination of prebiotics may be used such as 90% GOS with 10% short chain fructo-oligosaccharides such as the product sold under the trade mark Raftilose® or 10% inulin such as the product sold under the trade mark Raftiline®. A particularly preferred combination of prebiotics is 70% short chain fructo-oligosaccharides and 30% inulin.

The general composition of an infant formula according to the invention will now be described by way of example. The formula contains a protein source. The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in whatever proportions are desired.

The proteins may be intact or hydrolysed or a mixture of intact and hydrolysed proteins. It may be desirable to supply partially hydrolysed proteins (degree of hydrolysis between 2 and 20%), for example for infants believed to be at risk of developing cows' milk allergy. If hydrolysed proteins are required, the hydrolysis process may be carried out as desired and as is known in the art. For example, a whey protein hydrolysate may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

An infant formula according to the present invention contains a carbohydrate source. Any carbohydrate source conventionally found in infant formulae such as lactose, saccharose, maltodextrin, starch and mixtures thereof may be used although the preferred source of carbohydrates is lactose. Preferably the carbohydrate sources contribute between 35 and 65% of the total energy of the formula.

An infant formula according to the present invention contains a source of lipids. The lipid source may be any lipid or fat which is suitable for use in infant formulas. Preferred fat sources include palm olein, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils. In total, the fat content is preferably such as to contribute between 30 to 55% of the total energy of the formula. The fat source preferably has a ratio of n-6 to n-3 fatty acids of about 5:1 to about 15:1; for example about 8:1 to about 10:1.

The infant formula will also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form. The presence and amounts of specific minerals and other vitamins will vary depending on the intended infant population.

If necessary, the infant formula may contain emulsifiers and stabilisers such as soy lecithin, citric acid esters of mono- and di-glycerides, and the like.

The infant formula may optionally contain other substances which may have a beneficial effect such as lactoferrin, nucleotides, nucleosides, and the like.

Finally, the formula will contain Lactobacillus rhamnosus ATCC 53103 or Lactobacillus rhamnosus CGMCC 1.3724, Streptococcus salivarius DSM 13084 and Streptococcus thermophilus TH4® each in an amount between from 10⁴ to 10¹² cfu/g of composition (dry weight) and from 0.3 to 6% of a mixture of 90% GOS and 10% fructo-oligosaccharides.

The formula may be prepared in any suitable manner. For example, it may be prepared by blending together the protein, the carbohydrate source, and the fat source in appropriate proportions. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients. Commercially available liquefiers may be used to form the liquid mixture. The liquid mixture is then homogenised; for example in two stages.

The liquid mixture may then be thermally treated to reduce bacterial loads, by rapidly heating the liquid mixture to a temperature in the range of about 80° C. to about 150° C. for about 5 seconds to about 5 minutes, for example. This may be carried out by steam injection, autoclave or by heat exchanger; for example a plate heat exchanger.

Then, the liquid mixture may be cooled to about 60° C. to about 85° C.; for example by flash cooling. The liquid mixture may then be again homogenised; for example in two stages at about 10 MPa to about 30 MPa in the first stage and about 2 MPa to about 10 MPa in the second stage. The homogenised mixture may then be further cooled to add any heat sensitive components; such as vitamins and minerals. The pH and solids content of the homogenised mixture are conveniently adjusted at this point.

The homogenised mixture is transferred to a suitable drying apparatus such as a spray drier or freeze drier and converted to powder. The powder should have a moisture content of less than about 5% by weight.

The bacterial strains may be cultured according to any suitable method and prepared for addition to the dried infant formula by freeze-drying or spray-drying for example. Alternatively, bacterial preparations can be bought from specialist suppliers already prepared in a suitable form for addition to food products such as infant formula. The bacterial strains are added to the infant formula by dry mixing.

If a liquid product is preferred, the homogenised mixture may be sterilised then aseptically filled into suitable containers or may be first filled into the containers and then retorted. The bacterial strains will then be supplied separately ready to mix with the liquid at the point of consumption. For example the bacterial strains may be supplied packed in a separate sachet or in a separate compartment of the package containing the liquid.

In another embodiment, the composition may be a supplement including the strains in an amount sufficient to achieve the desired effect in an individual. This form of administration is more suited to children at the upper end of the target age group. Preferably the daily dose of the bacterial strains is between 10⁴ to 10¹² cfu. The amount of bacteria to be included in the supplement will be selected accordingly depending upon how the supplement is to be administered. For example, if the supplement is to be administered twice a day, each supplement will contain from 10² to 10⁶ cfu of each bacterial strain. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or enteral administration as well as vitamins, minerals trace elements and other micronutrients in accordance with the recommendations of Government bodies such as the USRDA.

### Example 1

An example of the composition of an infant formula according to the present invention is given below. This composition is given by way of illustration only.

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Prebiotic (70% FOS, 30% inulin) | 0.64 | 4.3 |
| (g) | | |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| I (μg) | 15 | 100 |
| Cu (mg) | 0.06 | 0.4 |
| Zn (mg) | 0.75 | 5 |

| | |
|---|---|
| L. rhamnosus ATCC 53103 | 2.10⁷ cfu/g of powder |
| S. salivarius DSM 13084 | 2.10⁷ cfu/g of powder |
| S. thermophilus TH4 | 2.10⁷ cfu/g of powder |

### Example 2

This example compares the inhibitory effect of a composition according to the invention on some of the pathogens commonly associated with otitis media with the effect of the constituents of the composition used alone. The bacterial strains tested were Lactobacillus rhamnosus ATCC 53103 and Streptococcus salivarius DSM 13084, alone and in combination. The pathogenic strains in respect of which inhibitory activity of the bacterial strains was assessed were three different strains of Streptococcus pneumoniae and one strain of Alloiococcus otitidis. The inhibitory spectra of the test strains were established by the use of deferred antagonism, as described previously (Tagg and Bannister 1979). Briefly, a 1 cm wise diametric streak culture of the test strain was inoculated onto the nutritionally appropriate agar. Following incubation, the macroscopic bacterial growth was removed with a glass slide and residual cells on the agar surface were killed by exposure to chloroform vapours for 30 minutes. The agar surface was the aired for 30 minutes. Todd Hewitt broth (THB, Difco) cultures (18 h, 37° C.) of the pathogenic strains were incoluared a right angles across the line of the original streak culture using swabs. After incubation for 24 hr at 37° C. in 5% carbon dioxide in air, the extent of inhibition of each pathogenic strain was measured. The results are shown in the Table below from which it may be seen that for all the pathogenic strains tested, the combination of Lactobacillus rhamnosus ATCC 53103 and Streptococcus salivarius DSM 13084 had a greater inhibitory effect than either of these strains used alone.

| | | TABLE | | | | | |
|---|---|---|---|---|---|---|---|
| | | Pathogen | | | | | |
| | | L. rhamnosus | S. salivarius | S. pneumoniae | S. pneumoniae | S. pneumoniae | A otitidis |
| | | ATCC 53103 | DSM 13084 | RX1 | D39 | TIGR4 | NZRCC 3648 |
| Inhibitor | | Origin | | | | | |
| strain | Media | R | R | R | R | R | R |
| 53103 | BACA | | 0 | 0 | 0 | 0 | 0 |
| 13804 | BACA | 0 | | 17 | 17 | 16 | |
| 53103/13804 | BACA | | | 20 | 18 | 18 | |
| 53103 | CHOC | | | | | | 0 |
| 13804 | CHOC | | | | | | 10 |
| 53103/13804 | CHOC | | | | | | 15 |

BACA Blood agar and calcium agar
CHOC Chocolate blood agar
Origin R = Reference Culture

1. A composition suitable for use in the prevention or treatment of otitis media comprising a non-pathogenic bacterial strain capable of increasing a systemic immune response, a non-pathogenic bacterial strain capable of exerting a bacteriostatic effect on pathogens associated with development of otitis media and a non-pathogenic bacterial strain capable of exerting a bacteriocidal effect on pathogens associated with development of otitis media.

2. A composition according to claim 1 wherein the bacterial strain capable of increasing the systemic immune response is selected from the group consisting of Lactobacillus rhamnosus ATCC 53103 and Lactobacillus rhamnosus CGMCC 1.3724.

3. A composition according to claim 1 wherein the bacteriocidal and bacteriostatic effects are exerted by a single bacterial strain.

4. A composition according to claim 3 wherein the single bacterial strain is selected from the group consisting of Micrococcus varians MCV8 and Streptococcus salivarius DSM 13084.

5. A composition according to claim 1 which further comprises Streptococcus thermophilus.

6. A composition according to claim 1 wherein the composition is a nutritional composition.

7. A composition according to claim 1 which comprises from 10⁴ to 10¹² cfu/g of composition (dry weight) of each strain.

8. A composition according to claim 7 which further comprises at least one prebiotic in an amount of from 0.3 to 6% by weight of the composition.

9. A composition according to claim 1 wherein the composition is an infant formula.

10. A composition according to claim 1 wherein the composition is a follow-on formula.

11. A composition according to claim 1 wherein the composition is a growing-up milk.

12. A composition according to claim 1 wherein the composition is a supplement and which comprises from 10⁴ to 10¹² cfu of each strain per unit dose.

13. A method for manufacturing a medicament or therapeutic nutritional composition for the prevention or treatment of otitis media comprising the steps of using a non-pathogenic bacterial strain capable of increasing a systemic immune response, a non-pathogenic bacterial strain capable of exerting a bacteriostatic effect on pathogens associated with development of otitis media and a non-pathogenic bacterial strain capable of exerting a bacteriocidal effect on pathogens associated with development of otitis media.

14. A method for the treatment of otitis media comprising the steps of administering to an individual having otitis media a therapeutically-effective amount of a composition comprising a non-pathogenic bacterial strain capable of exerting a bacteriostatic effect on pathogens associated with development of otitis media and a non-pathogenic bacterial strain capable of exerting a bacteriocidal effect on pathogens associated with development of otitis media.

15. A method according to claim 14 wherein the bacterial strain capable of increasing the systemic immune response is selected from the group consisting of Lactobacillus rhamnosus ATCC 53103 and Lactobacillus rhamnosus CGMCC 1.3724.

16. A method according to claim 14 wherein the bacteriocidal and bacteriostatic effects are exerted by a single bacterial strain.

17. A method according to claim 14 wherein the composition is a nutritional composition.

18. A method according to claim 14 which further comprises at least one prebiotic in an amount of from 0.3 to 6% by weight of the composition.
